(19) United States
(12) Patent Application Publication
(10) Pub. No.: US 2025/0037830 A1
(43) Pub. Date: Jan. 30, 2025

(54) ENERGY HARVESTING SUBSTANCE EVENT MONITORING SYSTEM

(71) Applicant: NOBLE INTERNATIONAL, LLC, Orlando, FL (US)

(72) Inventors: Jeffery A. LETTMAN, Orlando, FL (US); Paul SULLIVAN, Orlando, FL (US); Randy CARDOZA, Winter Garden, FL (US); Chris EVANS, Rehoboth Beach, DE (US)

(21) Appl. No.: 18/715,258

(22) PCT Filed: Dec. 1, 2022
(86) PCT No.: PCT/US22/51583

§ 371 Date: May 31, 2024

Related U.S. Application Data
(60) Provisional application No. 63/284,746, filed on Dec. 1, 2021.

(51) Int. Cl. G16H 20/17; G16H 40/67; H02J 50/00; H02J 50/05; H02J 50/10; H02J 50/20

(52) U.S. Cl. CPC G16H 20/17; G16H 40/67; H02J 50/001; H02J 50/05; H02J 50/10; H02J 50/20; H02J 2310/23

(57) ABSTRACT

An apparatus (800), including: an energy harvesting device (102, 804) configured to harvest energy and to convey the harvested energy; a communication device (102, 806); circuitry (104) configured to be powered by the harvested energy and to be in data communication with the communication device; an unpowered indicator (110) configured to exhibit an excursion indicator only when a temperature excursion of a substance (808) has occurred; and a registration sensor (112) configured to be powered by the harvested energy and when so powered and when the excursion indicator is present, to generate a registration of the excursion indicator. When powered by the harvested energy the circuitry is configured to transmit data comprising the registration of the excursion indicator via the communication device.

### FIELD OF THE INVENTION

The invention relates to an energy harvesting substance event monitoring system for a medicament delivery device.

### BACKGROUND

Studies of patients who are prescribed self-delivered biologic treatment show that the majority do not store their biologic disease-modifying drugs within the recommended temperature range. Inadequate storage of biopharmaceuticals may result in an ineffective therapeutic response.

In addition, as most biologics are stored at 2-8° C., they should be warmed to room temperature for about 30-45 min prior to administration to reduce or avoid any pain associated with the injection of a cold solution. Reducing injection associated pain by ensuring the medicament has warmed to an appropriate temperature before injection has been shown to increase adherence.

Further, adherence to systemic treatments decreases over time, with an overall adherence rate of 67% for injectable biologic medications. Medication non-adherence is associated with disease flares, increased disability, and increased costs. Median adherence in clinical studies as tracked using self-reported metrics has been shown to be grossly overestimated (17%). This demonstrates the need for Medication Event Monitoring Systems (MEMS) to obtain an accurate picture of adherence. Current medication event monitoring systems provide some help with the above, but there is room in the art for improvement.

### BRIEF DESCRIPTION OF THE DRAWINGS

The invention is explained in the following description in view of the drawings that show:

FIG. 1 is a block diagram representing an example embodiment of an energy harvesting medication event monitoring system in an operative relationship with an example embodiment of a medicament delivery device.

FIG. 2 is a block diagram of the energy harvesting medication event monitoring system and medicament delivery device of FIG. 1 in an operative relationship with an example embodiment of a medicament storage container.

FIG. 3 is a block diagram of the energy harvesting medication event monitoring system and medicament delivery device of FIG. 1 in an operative relationship with an example embodiment of a reusable device.

FIG. 4 shows an example embodiment of an NFC reader and an example embodiment of a graphic user interface.

FIG. 5 shows an example embodiment of the energy harvesting medication event monitoring system in an operative relationship with an example embodiment of a medicament delivery device.

FIG. 6 is a block diagram representing an example embodiment of an energy harvesting substance event monitoring system in an operative relationship with an example embodiment of a delivery device.

FIG. 7 is an embodiment of a flex circuit as described herein.

FIG. 8 is an embodiment of a device embodiment of a board incorporated into a grip on a medicament device.

### DETAILED DESCRIPTION

The present inventors have devised a unique and innovative energy harvesting substance event monitoring system that can gather information when dormant (i.e., unpowered), which can be energized by harvested energy, and which can report information gathered while dormant as well as information gathered while powered. In an example embodiment, the energy harvesting substance event monitoring system (EHSEMS) is configured to monitor a substance for a temperature excursion. The substance may be a medicament. In an example embodiment, the substance is a medicament disposed in a medicament delivery device. In another example embodiment, the substance may be a medicament substitute and the medicament substitute may be disposed in a medicament delivery device trainer/simulator. In an example embodiment, the medicament substitute may merely be any material or even simply the environment in which the energy harvesting substance event monitoring system is disposed. The medicament substitute could even be a component of the delivery device.

The term “delivery device” includes but is not limited to a training medicament delivery device or an actual medicament delivery device. This includes an injection device such as an auto-injector and/or trainer, an inhaler device and/or respiratory trainer, a bolus injector, a nasal inhaler device, a needless injector, a transdermal patch, an ampoule, a medicine container, a medicine package, a vial, a metered-dose inhaler, a dry powder inhaler, a prefilled syringe, among other medicament devices known to one of ordinary skill in the art.

In an example embodiment, the energy harvesting substance event monitoring system is configured to harvest energy using an energy harvesting device. In an example embodiment, the energy harvesting device harvests energy via an energy field. An example of such an energy harvesting device is an antenna of a near field communication (NFC) system. In an alternate example embodiment, the energy harvesting device harvests energy of mechanical movement. In another alternate example embodiment, the energy harvesting device harvests energy through heat exchange. In alternate example embodiments, the energy harvesting device can use any other known energy harvesting technology to harvest energy. Any number and any combination of these energy harvesting devices may be present in a single energy harvesting substance event monitoring system.

In an example embodiment, the energy harvesting substance event monitoring system is configured to communicate data to an external device via a communication device. In an example embodiment, the communication device is configured to be connected to the external device via wireless communication. Example wireless communication standards that can be used include Wi-Fi, Bluetooth®, and near field communication (NFC). In an example embodiment, the communication device is powered by the harvested energy. In an alternate example embodiment, the communication device is configured to be connected to the external device via a wired connection. A wireless communication device (e.g., an antenna, a transmitter) can be powered by the harvested energy, as can a wired communication device (e.g., Bluetooth®, WiFi, NFC).

In an example embodiment, both the energy harvesting function and the wireless communication function are achieved using an NFC communication system/protocol. In an NFC system, an external device (an NFC reader) delivers energy to the energy harvesting substance event monitoring system which is set up to harvest and utilize the delivered energy. Additionally, the NFC reader and the energy harvesting substance event monitoring system communication device communicate via NFC standards. In an example embodiment, the NFC reader is a smartphone.

The example embodiments disclosed herein are suitable for use with a substance delivery device with a medicament therein or a substance delivery device trainer/simulator with a substance therein. In the latter, the substance may be selected to have thermal characteristics similar to those of a medicament. Alternately, the substance can be any substance (e.g., liquid, solid, gel etc.) that reflects the temperature in which the delivery device is disposed. For discussion purposes, the first example embodiment discussed below relates to a substance delivery device with medicament therein, namely a medicament delivery device. However, the delivery device could also be a delivery device trainer/simulator.

In the example embodiments disclosed herein, a passive (i.e., unpowered) sensor is in thermal communication with substance and is configured to undergo a registrable change if the substance experiences a temperature excursion beyond a threshold at any time during the thermal communication. A powered registration sensor in the device is powered by the energy harvested by the device. Once powered by the harvested energy, the powered registration sensor is configured to generate a registration if the passive sensor has undergone the registrable change associated with the temperature excursion of the substance and to report the registration, if present, to the NFC reader. The device may also include one or more other sensors. In an example embodiment, this includes a powered temperature sensor configured to be powered by the harvested energy, to measure a current temperature of the substance, and to report the current temperature to the NFC reader. The passive and active temperature collection will provide the user with some feedback regarding the substance storage conditions and resulting efficacy of a medicament so stored, regarding the current temperature of the substance and resulting injection comfort, and regarding an adherence which is inferred by the powering-up of the device and associated expectation that this is done prior to an actual injection.

### Detailed Description of Illustrated Embodiments

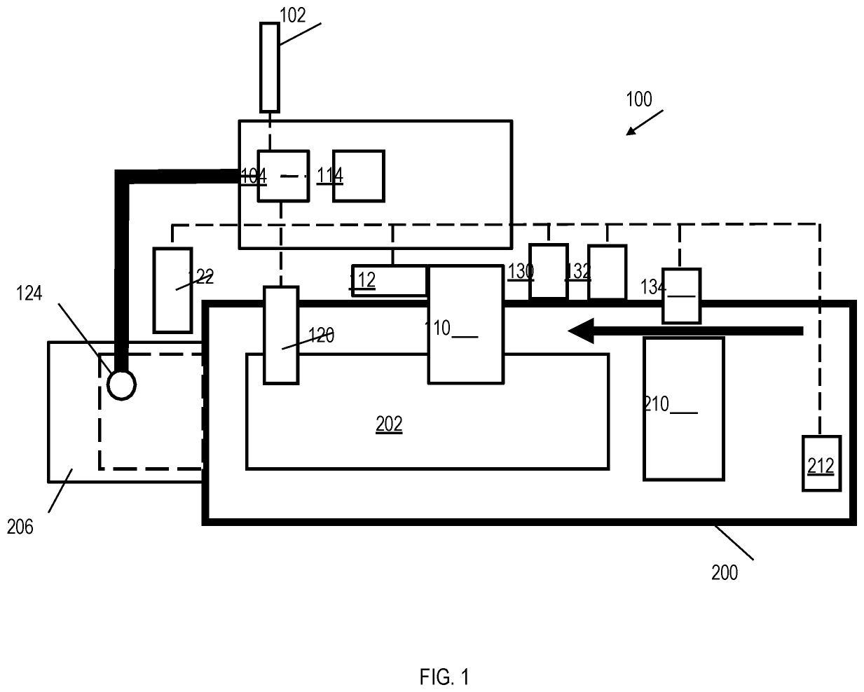

FIG. 1 is a block diagram representing an example embodiment of an energy harvesting substance event monitoring system (EHSEMS). Specifically, FIG. 1 discloses energy harvesting medication event monitoring system (EHMEMS) 100 in an operative relationship with an example embodiment of a medicament delivery device 200 that contains medicament 202. The EHMEMS 100 includes an NFC antenna 102 configured to harvest energy and when powered by the harvested energy, to convey the harvested energy to energy consuming components of the EHMEMS 100. The EHMEMS 100 further includes circuitry 104 configured to be powered by the harvested energy and to be in data communication with the NFC antenna 102. Such data communication includes transmitting data to and receiving data from the NFC reader. The NFC antenna 102 and the circuitry 104 can use any energy harvesting technology. In an example embodiment, the energy harvesting technology is based on NFC protocols known to the artisan.

The EHMEMS 100 further includes an unpowered/passive indicator 110 that is configured to indicate when a temperature excursion of the medicament 202 in the medicament delivery device 200 has occurred. Such an unpowered indicator 110 may be a physically or chemically activated indicator/sensor that includes a physical substance or a chemical that undergoes a registrable change during the temperature excursion. The indication may be the change in chemical composition that results in, for example, a color change in the unpowered indicator 110. However, a variety of other ways to generate a registrable change during and/or as a result of a temperature change. This includes using a material: that has a melting point temperature above a predetermined temperature associated with the temperature excursion; that undergoes an enzyme reaction above a predetermined temperature associated with the temperature excursion; that polymerizes above a predetermined temperature associated with the temperature excursion; that corrodes above a predetermined temperature associated with the temperature excursion; that forms liquid crystals above a predetermined temperature associated with the temperature excursion; and/or that undergoes an irreversible microbiological change above a predetermined temperature associated with the temperature excursion. The change in color is therefore an example of a registrable excursion indicator and happens only when the temperature exceeds a predetermined threshold. The unpowered indicator 110 is therefore able to indicate when a temperature of the medicament 202 has exceeded a threshold regardless of whether the EHMEMS 100 is powered. An example of a suitable unpowered indicator 110 is a cold chain indicator that provides a physical indication, such as a WarmMark temperature indicator label/sticker manufactured by SpotSee of Dallas TX. In addition to color change, motion is another indication method used by some time-temperature sensors.

In at least one specific, non-limiting embodiment, the unpowered/passive indicator turns from a first color to a second color (i.e., white to red) when an excursion event has occurred. When powered via NFC energy harvesting, the board shines a green LED light at this indicator. This light is reflected onto a green light sensor on the board. When the indicator is white, the green light is reflected, resulting in a high reading from the light sensor. When the indicator turns red, green light is absorbed and less is reflected back to the sensor.

In at least one non-limiting embodiment, as an alternative to measuring the temperature of a substance (i.e., medication), an embodiment of the device may be attached to a mass simulator. The mass simulator would be designed to respond to temperature in a manner similar to the medicament in the device.

For example, knowing the medicament is 1 mL in an autoinjector. A temperature study may be conducted to show that 10 grams of aluminum transitions at the same rate. In one non-limiting embodiment, as an alternative to applying the NFC device directly to the autoinjector, the device can be attached to an aluminum mass simulator. In one non-limiting example, the aluminum mass simulator may be in the shape of a business card. This card shaped device can be stored with the medicament in the fridge. When ready to inject, both the card and device can be placed in a room temperature environment to warm up. This provides one embodiment of a reusable version of the device, as the card would be retained, rather than disposing of it along with the auto-injector after injection.

The EHMEMS 100 further includes a registration sensor 112 configured to be powered by the harvested energy. When powered by the harvested energy, the registration sensor 112 is configured to detect the registrable excursion indicator (e.g., the color change) of the unpowered indicator 110. If the excursion indicator is present, the registration sensor 112 is further configured to indicate the presence of the excursion indicator by generating a registration of the excursion indicator. The circuitry 104 is in data communication with the registration sensor 112 so the circuitry 104 is informed of the registration of the excursion indicator, which registration may be included in the data transmitted by the EHMEMS 100 to the NFC reader. Hence, if a temperature excursion of the medicament 202 has occurred (e.g., during storage when the EHMEMS is unpowered), the EHMEMS 100 will indicate the occurrence once powered by the NFC reader. An example of a suitable registration sensor 112 includes photoresistors and colors sensors. Mechanical changes could be detected via an associated change in electrical state (e.g., a switch).

The EHMEMS 100 optionally further includes a memory device 114 (e.g., a digital memory device) that is configured to be powered by the harvested energy and to store data. Stored data can include data indicating that the registration sensor 112 has indicated the presence of the temperature excursion. The stored data can also include data pertaining to the medicament 202 and any other data associated with the EHMEMS 100 and the medicament delivery device 200. The circuitry 104 is in data communication with the memory device 114 so the circuitry 104 can convey data to and receive data from the memory device 114. As such, data transmitted to the NFC reader can include any data present in the memory device 114. In an example embodiment, the circuitry 104 may include System on Chip (SoC) with Electrically Erasable Programmable Read Only Memory (EEPROM). In addition to memory device 114, the SoC may include a digital unit controller and interface (typically I2C) and optionally a voltage regulator and analog front end.

The EHMEMS 100 optionally further includes a temperature sensor 120 configured to be powered by the harvested energy. When powered by the harvested energy, the temperature sensor 120 is configured to measure a current temperature of the medicament 202. The circuitry 104 is in data communication with the temperature sensor 120 so the circuitry 104 receives the current temperature data, which may be included in the data transmitted by the EHMEMS 100 to the NFC reader. Examples of suitable temperature sensor 120 include resistance temperature detectors (RTD), thermistor, thermocouples, or IC sensors like a polymer dielectric planar capacitor.

The EHMEMS 100 optionally further includes an injection sensor 122 configured to be powered by the harvested energy. When powered by the harvested energy, the injection sensor 122 is configured to detect a wet injection. If the injection sensor 122 detects a wet injection, the injection sensor 122 is configured to generate a registration of the wet injection. The circuitry 104 is in data communication with the injection sensor 122 so the circuitry 104 receives the registration of wet injection, which may be included in the data transmitted by the EHMEMS 100 to the NFC reader. Wet injection can be detected directly via moisture/humidity sensors or via a MEMS (microelectromechanical system) chemical sensor or biochemical sensor to detect the medicament.

The EHMEMS 100 optionally further includes a displacement sensor 124 configured to be powered by the harvested energy. When the displacement sensor 124 is powered by the harvested energy, the displacement sensor 124 is configured to detect a displacement of the displacement sensor 124. If the displacement sensor 124 detects displacement of the displacement sensor 124, the displacement sensor 124 is configured to generate a registration of the displacement. The circuitry 104 is in data communication with the displacement sensor 124 so the circuitry 104 receives the registration of displacement of the displacement sensor 124, which may be included in the data transmitted by the EHMEMS 100 to the NFC reader. In an example embodiment, the displacement sensor 124 is associated with a cap 206 of the medicament delivery device 200. Hence, if the cap 206 is moved/removed, the displacement sensor 124 will indicate this. A MEMS (microelectromechanical system) accelerometer or IMU could be used as a displacement sensor 124 to recognize movement characteristic of device usage (i.e. cap removal, device activation, device movement).

The EHMEMS 100 optionally further includes a pressure sensor 130 configured to be powered by the harvested energy. When the pressure sensor 130 is powered by the harvested energy, the pressure sensor 130 is configured to detect a pressure associated with the medicament 202 and/or the medicament delivery device 200. Such pressure may indicate activation of the medicament delivery device 200. The circuitry 104 is in data communication with the pressure sensor 130 so the circuitry 104 receives the pressure data, which may be included in the data transmitted by the EHMEMS 100 to the NFC reader. An example of a suitable pressure sensor 130 includes MEMS pressure sensors like a piezoresistive absolute pressure sensor or thin-film pressure sensors.

The EHMEMS 100 optionally further includes an acceleration sensor 132 configured to be powered by the harvested energy. When the acceleration sensor 132 is powered by the harvested energy, the acceleration sensor 132 is configured to detect an acceleration associated with the medicament 202 and/or the medicament delivery device 200. Such acceleration may indicate motion of any sort, such as removal of the cap 206 or activation of the medicament delivery device 200. The circuitry 104 is in data communication with the acceleration sensor 132 so the circuitry 104 receives the acceleration data, which may be included in the data transmitted by the EHMEMS 100 to the NFC reader. A suitable acceleration sensor 132 could be an accelerometer or inertial measurement unit sensors.

The EHMEMS 100 optionally further includes a second displacement sensor 134 configured to be powered by the harvested energy. When the second displacement sensor 134 is powered by the harvested energy, the second displacement sensor 134 is configured to detect a second displacement associated with the medicament 202 and/or the medicament delivery device 200. The circuitry 104 is in data communication with the second displacement sensor 134 so the circuitry 104 receives the second displacement data, which may be included in the data transmitted by the EHMEMS 100 to the NFC reader. A suitable second displacement sensor 134 can be an accelerometer, an inertial measurement unit sensor, a strain sensor, and/or a severable portion of an electrical circuit.

In addition to detecting movement of the overall device via an accelerometer, one could also detect movement of individual components within the device. In an example embodiment, the second displacement sensor 134 may be associated with an individual movable component 210 of the medicament delivery device 200. In an example embodiment, the movable component 210 may be a plunger or the like that moves the medicament 202 through an injection needle as the movable component 210 moves. Movement of the movable component 210 may be used to imply a dispensing of the medicament 202 (i.e., activation of the medicament delivery device 200) and an amount of the movement may be used to imply an amount of the medicament 202 dispensed. From the data, one may be able to infer a dose of the medicament 202 actually received by the patient. However, the dosage may be reduced if the injection sensor 122 registers a wet injection, which would mean that at least some of the medicament 202 was not properly delivered to the patient.

In an alternate example embodiment, detecting removal of the cap 206 can be accomplished via a strain sensor attached between a relatively immovable part of the medicament delivery device 200 (e.g., a body) and cap 206. Alternately, detecting removal of the cap 206 can be accomplished via a breakable trace in the circuit that spans the relatively immovable part and cap 206, so that when the cap 206 is removed, the loop in the circuit is severed. In yet another alternate, a switch could be used to sense this displacement if placed so that removal of the cap 206 or activation of the medicament delivery device 200 causes the switch to change state.

The EHMEMS 100 optionally further includes a smart metamaterial 212. In an example embodiment, the smart metamaterial can be employed to perform in any of the sensing activities disclosed above (pressure, strain, biochemical etc.) or as an alternative to a PCB NFC antenna design (while still interfacing via standard NFC protocols). The smart metamaterial 212 may be configured to be powered by the harvested energy. The circuitry 104 is in data communication with the smart metamaterial 212 so the circuitry 104 receives the smart metamaterial data, which may be included in the data transmitted by the EHMEMS 100 to the NFC reader.

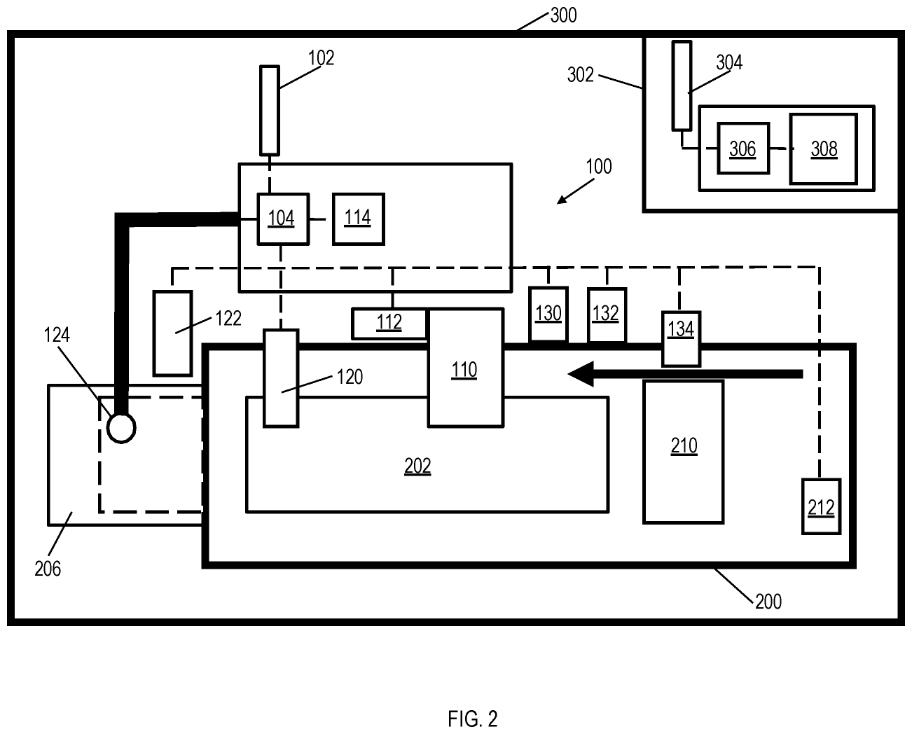

FIG. 2 is a block diagram of the EHMEMS 100 and the medicament delivery device 200 of FIG. 1 in an operative relationship with an example embodiment of a medicament storage container 300. In this example embodiment, the medicament storage container 300 includes an NFC reader 302 including an NFC reader antenna 304, reader circuitry 306, and reader memory 308 in data communication with the reader circuitry 306. In this example embodiment, the NFC reader 302 is configured to provide energy for the NFC antenna 102 to harvest. The temperature sensor 120 is configured to take plural measurements of the current temperature of the medicament 202 over time while the NFC reader 302 provides the energy. The data transmitted by the circuitry 104 includes data related to the plural measurements of the current temperature of the medicament 202. The data related to the plural measurements may be stored in the reader memory 308 to be read by a further/external NFC reader or may be accessed in any suitable way known to the artisan. Hence, when disposed in the medicament storage container 300 and powered by the NFC reader 302, the EHMEMS 100 can provide constant monitoring of the temperature of the medicament 202.

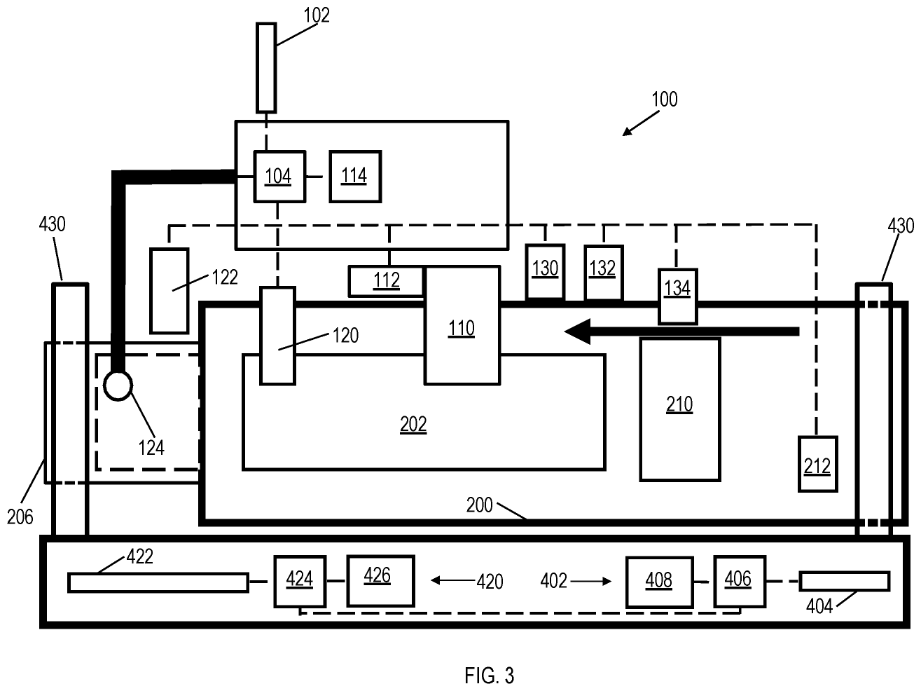

FIG. 3 is a block diagram of the EHMEMS 100 and medicament delivery device 200 of FIG. 1 in an operative relationship with an example embodiment of a reusable device 400. In this example embodiment, the reusable device 400 includes a reusable device NFC reader 402 including a reusable device NFC antenna 404, reusable device circuitry 406, and reusable device memory 408 in data communication with the reusable device circuitry 406. The reusable device 400 also includes a reusable device data communicator 420 with a reusable device data communicator antenna 422, reusable device data communicator circuitry 424, and reusable device data communicator memory 426. The reusable device data communicator 420 is in wired and/or wireless data communication with the reusable device NFC reader 402. The reusable device data communicator 420 is also configured to operate using Bluetooth® technology or Wi-Fi technology to communicate data with a further/external data device. Such an arrangement can extend a range the data from the circuitry 104 can be transmitted from that associated with NFC to that associated with Wi-Fi and/or Bluetooth® etc. An example of a suitable reusable device data communicator circuitry 424 uses an SoC (system on chip) specifically designed for low-power Bluetooth communication (BLE) as is described in U.S. Pat. No. 9,767,708 to Baker et al.

An example reusable device 400 includes an injection aid configured to properly position/guide the medicament delivery device 200 via e.g., guides 430. In certain instance, a single reusable device 400 may be associated with the use of plural different medicament delivery devices 200 over time. In such a scenario, the reusable device 400 can collect data from each of the medicament delivery devices 200 by virtue of having the incorporated reusable device NFC reader 402. The reusable device 400 can store the collected data in the reusable device data communicator memory 426 for immediate transmission to the further/external data device and/or the collected data can be stored for later transmission to the further/external data device. It is also possible that the further/external data device can be a device that is periodically or constantly connected to the internet, such as a router. In such a scenario, the reusable device 400 can be in periodic or constant data communication over the internet with a remote data device that can store and/or use the data in any way deemed suitable.

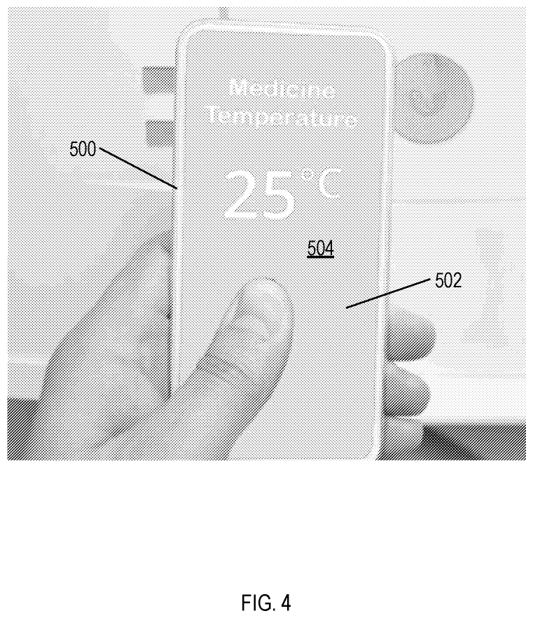

FIG. 4 shows an example embodiment of an NFC reader 500 and an example embodiment of a graphic user interface 502 displayed thereon. In this example embodiment, the NFC reader is a smart device (e.g., a smartphone, tablet, smartwatch, smart glasses or other personal electronic device) and the graphic user interface 502 displays the current temperature of the medicament. In an example embodiment, the graphic user interface 502 can display a background 504 in one color to indicate no registration of the temperature excursion (e.g., a green background), and can display the background 504 in a different color to indicate registration of the temperature excursion (e.g., a red background). Alternately, the graphic user interface 502 can display an icon or otherwise indicate whether the temperature excursion has been registered.

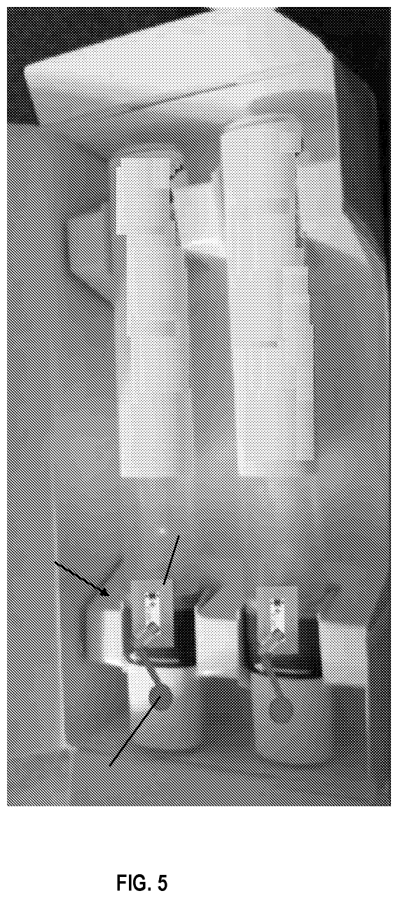

FIG. 5 shows an example embodiment of an EHMEMS 600 in an operative relationship with an example embodiment of a medicament delivery device 700. The EHMEMS 600 can be applied to the medicament deliver device 700 prior to receipt by a patient. This EHMEMS 600 provides at least: temperature excursion detection during storage; temperature measurement prior to injection, to minimize injection site pain; and communication of adherence data (injection date, time, location, medicament information, and storage information) via secure communication with a patient's NFC reader (e.g., smartphone). By building in value adding functionality for the patient (temperature at time of injection), the system would be better positioned to collect reliable adherence data. Since the EHMEMS 600 leverages energy harvesting technologies (NFC), it would not require a power source, and could be very low cost.

When ready for injection, the patient can use the EHMEMS 600 to receive confirmation that the medicament is at the optimal temperature for injection. This helps minimize any pain due to the medication being below an appropriate temperature for injection. The NFC communication does not require pairing with the EHMEMS 600. Bringing the smartphone within range (˜10 cm) is all that would be required. This provides additional incentive for the patient to interact with the EHMEMS 600 (beyond adherence tracking). With measured confirmation that the medication has reached room temperature, the interaction could also serve as an effective medication event monitor by inferring the injection has been administered after the EHMEMS 600 has been read by the NFC reader. The EHMEMS 600 can also store information about the medicament, helping to ensure proper dosing. Since this is completed in an unpowered device (harvesting energy from the reader), it could be extremely low cost.

The EHMEMS 600 includes a tag 602 that is secured to the medicament deliver device 700. The tag 602 includes at least some (or all) of the components disclosed above as being part of the EHMEMS 100. Also visible is the displacement sensor 604 which is secured to the cap 702 of the medicament deliver device 700 and which will register displacement associated with removal of the cap 702 when read by the NFC reader.

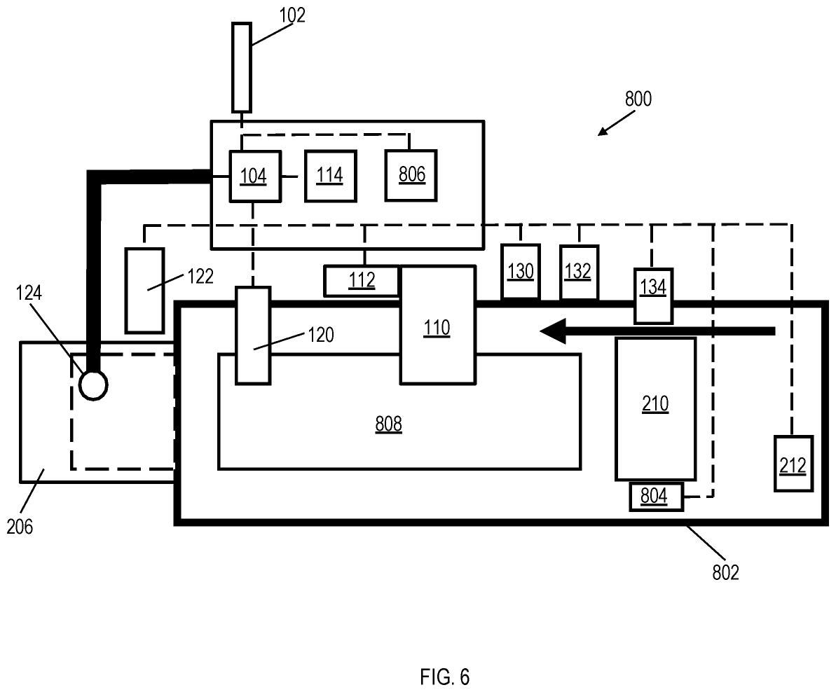

FIG. 6 is a block diagram representing an example embodiment of an energy harvesting substance (as opposed to medicament) event monitoring system (EHSEMS) 800 in an operative relationship with an example embodiment of a delivery device 802. This alternate example embodiment of the EHSEMS 800 is similar to the EHMEMS 100 of FIG. 1, except that the EHSEMS 800 includes an energy harvesting device 804 and a communication device 806. The energy harvesting device 804 may be any type of energy harvesting device. The energy harvesting device 804 may be a duplicate/redundant NFC energy harvesting device. Alternately, the energy harvesting device 804 may be any type of energy harvesting device other than an NFC energy harvesting device. Energy harvesting devices other than NFC include, but are not limited to, mechanical energy harvesting devices and heat transfer energy harvesting devices.

The delivery device 802 shown is similar to the medicament delivery device 200 of FIG. 1, except that the delivery device 802 contains a substance 808 that may or may not be medicament. Also, the delivery device 802 may be a medicament delivery device or a medicament delivery device trainer.

The EHSEMS 800 shows the NFC antenna 102 together with the energy harvesting device 804 and the communication device 806. It is also possible to have either the energy harvesting device 804 or the communication device 806 present instead of both. It is also possible to dispense with the NFC antenna 102 entirely.

Hence, any combination of the NFC antenna 102, the energy harvesting device 804 (or energy harvesting devices 804), and the communication device 806 (or communication devices 806) is possible.

The NFC antenna 102 provides two functions. One function is to harvest energy. Another function is to communicate data. Hence, the NFC antenna 102 is both an energy harvesting device and a communication device. The energy harvesting function can be supplemented with or replaced by the energy harvesting function of the energy harvesting device 804. Similarly, the data communication function can be supplemented with or replaced by the data communication function provided by the communication device 806.

One or more energy harvesting devices 804 can be associated with (e.g., connected to) one or more components in the delivery device 802 that undergo movement. For example, the when the energy harvesting device 804 is a mechanical energy harvesting device, the energy harvesting device 804 can be associated with the movable component 210. In such a configuration, when the movable component 210 (e.g., the plunger) is depressed, the energy harvesting device 804 can harvest energy which can be used to supplement or fully power the EHSEMS 800.

Alternately, or in addition, the one or more energy harvesting devices 804 can include mechanical energy harvesting devices configured to harvest energy from other motion. Other motion includes motion that the energy harvesting device 804 experiences during activities such as transportation. In such an example embodiment, transportation movement will enable the energy harvesting device 804 to harvest energy and power the EHSEMS 800 during transportation. Data can then be acquired via the various sensors during transportation and stored in the memory device 114 for later retrieval. The later retrieval can be accomplished by providing energy for the same energy harvesting device 804, or by providing energy for an alternate/secondary energy harvesting device to retrieve. The alternate/secondary energy harvesting device can be a mechanical energy harvesting device 804 that harvests energy from a different type of motion, such as that of the movable component 210, and/or an NFC antenna 102.

Mechanical energy harvesting can be divided into two main groups, kinetic energy due to rigid body motion and elastic energy due to elastic deformation. Example types of mechanical energy harvesting devices include: electromagnetic energy harvesting (relative motion between permanent magnets (PMs) and conductor coils); electrostatic energy harvesting (current generation from variable capacitance induced by mechanical vibration); piezoelectric energy harvesting (generation of electric polarization due to the applied mechanical stress or the mechanical strain induced by the applied electric field); and triboelectric energy harvesting (rely on the electrification of a given material when it comes into frictional contact with a different material).

Energy harvesting can also be achieved through heat exchange (heat exchange energy harvesting devices) using thermoelectric energy harvesters (TEEHs) or pyroelectric energy harvesters (PEEHs). TEEHs are built on the principle of thermoelectric conversion describing the heat flow induced by spatial temperature difference and electricity. The pyroelectric effect characterizes the temporary generation of electricity due to the orientation change of spontaneous polarization caused by temperature fluctuations. Heat exchange energy harvesting devices could potentially harvest the energy necessary (e.g., for the communication device 806 etc.) from the process of bringing the delivery device 802, the substance 808, and/or the EHSEMS 800 to room temperature from refrigerated storage.

Likewise, one or more communication devices 806 can be present whether or not the NFC antenna 102 is present. If the communication device 806 requires harvested energy, the communication device 806 may be configured to receive energy from the energy harvesting device 804 and/or the NFC antenna 102.

The one or more communication devices 806 can provide data communication in one or more formats. For example, the communication device 806 can be a duplicative NFC communication device. Alternately, or in addition, the communication device 806 can communication in other wireless formats (e.g., Wi-Fi, Bluetooth®), and/or may communicate via a wired connection. In this way, a variety of communication options can be presented in one EHSEMS 800.

In an example embodiment without the NFC antenna 102, energy can be harvested via the energy harvesting device 804 which can be used, in turn, to power the EHSEMS 800. The sensors can retrieve data and the data can be transmitted via the communication device 806 wirelessly and/or via a wired connection.

Regarding operation, the EHSEMS 800 may: maintain a thermal association between a substance 808 and an unpowered indicator 110, wherein a property of the unpowered indicator 110 changes if the unpowered indicator 110 exceeds a predetermined temperature; maintain an association between a registration sensor 112 and the unpowered indicator 110, wherein the registration sensor 112 is configured to be powered by harvested energy and when so powered and when the excursion indicator is present, to generate a registration of the excursion indicator; and maintain an association between an energy harvesting device and the registration sensor 112, wherein the energy harvesting device 102, 804 is configured to harvest energy and to deliver harvested energy to the registration sensor 112 when the energy harvesting device 102, 804 harvests energy.

The EHSEMS 800 may further: harvest energy via the energy harvesting device 102, 804; provide the harvested energy to the registration sensor 112; determine whether the excursion indicator is present via the registration sensor 112; and generate a registration of the excursion indicator via the registration sensor 112 when the excursion indicator is present and when the registration sensor 112 is powered by the energy harvesting device 102, 804.

The EHSEMS 800 may further: establish data communication between the registration sensor 112 and a communication device 102, 806 configured to be powered by the harvested energy when the registration sensor 112 and the communication device 102, 806 are powered by the harvested energy; and transmit data via the communication device 102, 806 when the communication device 102, 806 is powered by the harvested energy, wherein the data comprises whether the registration is present.

The EHSEMS 800 may further: provide thermal communication between the substance 808 and a temperature sensor 120 that is configured to be powered by the harvested energy; measure a temperature of the substance 808 via the temperature sensor 120 when the temperature sensor 120 is powered by the harvested energy; and establish data communication between the temperature sensor 120 and the communication device 102, 806; wherein the data comprises the temperature of the substance 808.

The EHSEMS 800 may infer an activation of the device at a date and a time the energy harvesting device 102, 804 harvests the energy. The EHSEMS 800 may determine a degree of adherence to an activation plan (e.g., an administration of medication plan/schedule, or an administration of simulated medication plan/schedule) based at least in part on the inference.

The EHSEMS 800 may receive energy from a smart device 500. The smart device 500 may provide information when the energy is delivered to the energy harvesting device 102, 804, wherein the information comprises training materials related to the method. The smart device 500 may share the data with an authorized user via the internet. Authorized users may include any or all of health care providers, pharmacies, and any other authorized users deemed suitable.

A user operating the EHSEMS 800 may remove a delivery device comprising a substance 808 from cold storage; warm the substance 808; establish communication with an energy harvesting substance event monitoring system associated with the delivery device by providing energy for an energy harvesting device 102, 804 in the energy harvesting substance event monitoring system; and capture data from the energy harvesting substance event monitoring system, wherein the data comprises whether or not a passive sensor in the energy harvesting substance event monitoring system indicates a temperature excursion of the substance 808 has occurred.

A user may then determine whether a temperature of the substance 808 is at or above a predetermined temperature via a temperature sensor 120 in the energy harvesting substance event monitoring system.

A user may then infer an activation of the device at a date and a time the energy harvesting device 102, 804 harvests the energy. The user may determine a degree of adherence to an activation plan based at least in part on the inference

The smart device 500 may be used to at least one of: provide the energy; establish the communication; and to capture the data. A user may then review training resources presented on the smart device 500 once the communication with the energy harvesting substance event monitoring system is established. The EHSEMS 800 shares the data from the energy harvesting substance event monitoring system with an authorized user. The sharing may occur over the internet.

The energy harvesting medication event monitoring system disclosed herein can temperature excursions, along with other data, when powered. The energy harvesting medication event monitoring system harvests the energy necessary to report the information and therefore requires no power source. This allows for low-cost production of a device that can have a positive impact on people's lives in a way not previously known. Consequently, the energy harvesting medication event monitoring system represents an improvement in the art.

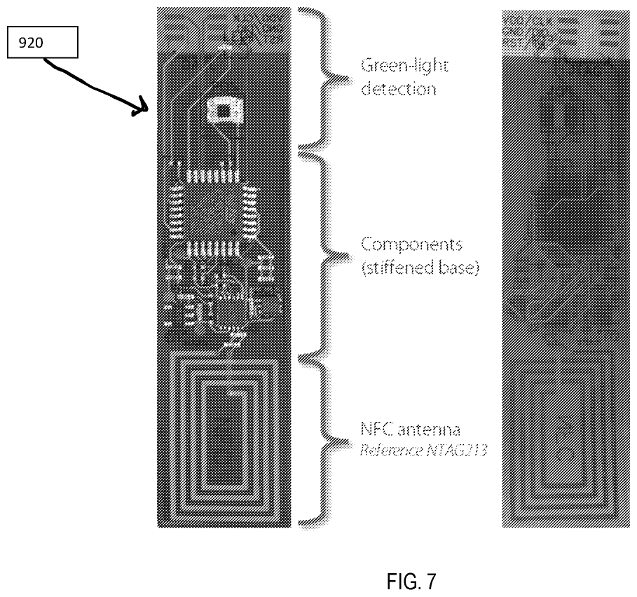

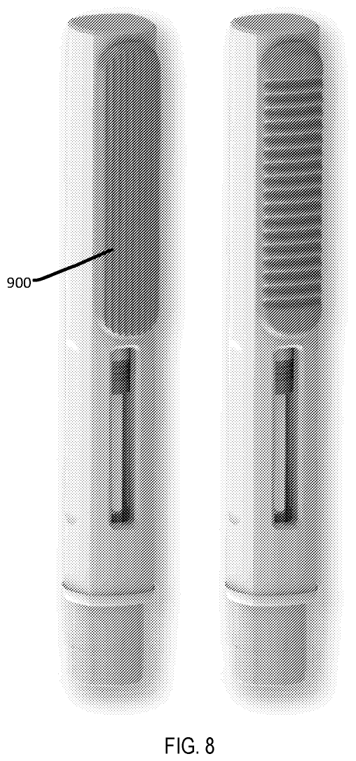

In at least some non-limiting embodiments, the PCB and/or NFC circuit may be provided on a flex (or flexible) circuit. An embodiment of a flex circuit 920 is shown in FIG. 7 showing the green light detection portion, the base, and the NFC antenna. Use of a flex circuit provides flexibility in the size of the device housing the PCB and/or NFC. The flex circuit allows the size of the device to be decreased and more compact in at least one embodiment. In at least one embodiment, the board and/or NFC circuit may be configured such that it may be applied to a device such as an autoinjector in the form of a label. In a further embodiment, the data transmitted via NFC may include manufacturing information, manufacturing date, lot number, trackability information and storage information, or other data relating to the device, the use of the device, or the user. In a further embodiment, the apparatus may be comprised in a medicament device attachment component, said attachment component 800 configured for attaching to a medicament device. In still a further embodiment, the attachment component 800 may include a rubber and/or silicone material, configured to attach to the medicament device to provide a grip for the medicament device as shown in FIG. 8. The grip allows a user to manipulate the medicament device while maintaining a firm grasp on the medicament device. The grip prevents slipping of the fingers of the user during use of the medicament device.

All features disclosed in the specification, including the claims, abstract, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent, or similar purpose, unless expressly stated otherwise.

While various embodiments of the present invention have been shown and described herein, it will be obvious that such embodiments are provided by way of example only. Numerous variations, changes and substitutions may be made without departing from the invention herein. Accordingly, it is intended that the invention be limited only by the spirit and scope of the appended claims.

The invention claimed is:

1. An apparatus, comprising:
an NFC antenna configured to harvest energy and when powered by harvested energy to convey the harvested energy;
circuitry configured to be powered by the harvested energy and to be in data communication with the NFC antenna;
an unpowered indicator configured to exhibit an excursion indicator only when a temperature excursion of a substance has occurred; and
a registration sensor configured to be powered by the harvested energy and when so powered and when the excursion indicator is present, to generate a registration of the excursion indicator;
wherein when powered by the harvested energy the circuitry is configured to transmit data comprising the registration of the excursion indicator via the NFC antenna.

2. The apparatus of claim 1, further comprising a memory device that when powered by the harvested energy is configured to store data indicating that the registration sensor has indicated a presence of the temperature excursion.

3. The apparatus of claim 1, further comprising a memory device configured to be powered by the harvested energy and to store data.

4. The apparatus of any of claims 1-3, wherein the unpowered indicator comprises a chemically activated sensor comprising a chemical that undergoes a change in chemical composition during the temperature excursion.

5. The apparatus of claim 4, wherein the change in chemical composition results in a color change.

6. The apparatus of any of claims 1-5, further comprising a temperature sensor configured to be powered by the harvested energy and when so powered is configured to measure a current temperature of the substance; wherein the data transmitted comprises the current temperature.

7. The apparatus of any of claims 1-6, wherein the NFC antenna is configured to transmit data via NFC communication protocols.

8. The apparatus of any of claims 1-7, further comprising a delivery device comprising the substance.

9. The apparatus of claim 8, wherein the delivery device comprises a medicament delivery device and the substance comprises a medicament.

10. The apparatus of claim 8, wherein the delivery device comprises a medicament delivery device trainer.

11. The apparatus of any of claims 1-10, further comprising an injection sensor: configured to be powered by the harvested energy; configured when so powered to detect a wet injection; and configured to generate a registration of the wet injection when the wet injection has been detected; and wherein the data transmitted comprises the registration of the wet injection.

12. The apparatus of any of claims 1-11, further comprising a displacement sensor: configured to be powered by the harvested energy; configured when so powered to detect a displacement of the displacement sensor; and
configured to generate a registration of the displacement when the displacement has been detected; and wherein the data transmitted comprises the registration of the displacement.

13. The apparatus of claim 12, wherein the displacement sensor is associated with a cap of a delivery device comprising the substance.

14. The apparatus of claim 6, further comprising:
an NFC reader configured to provide energy for the NFC antenna to harvest;
wherein the temperature sensor is configured to take plural measurements of the current temperature of the substance when the NFC reader provides the energy;
wherein the data transmitted by the circuitry comprises the plural measurements of the current temperature of the substance; and
wherein the NFC reader is configured to receive and store data comprising the plural measurements of the current temperature, wherein the substance is optionally a surface of a medicament device, a medicament and/or a mass simulator indicative of a temperature of the medicament.

15. The apparatus of claim 14, wherein the NFC reader is associated with a storage container configured to store a delivery device comprising the substance.

16. The apparatus of claim 8, further comprising a reusable device configured to operatively interact with the delivery device;
wherein the reusable device comprises an NFC reader configured to provide energy for the NFC antenna to harvest when the delivery device is operatively interacting with the reusable device; and
wherein the NFC reader is configured to receive the data transmitted by the circuitry.

17. The apparatus of claim 16, wherein the reusable device further comprises a reusable device data communicator that is in data communication with the NFC reader and also configured to operate using Bluetooth® technology or Wi-Fi technology.

18. The apparatus of claim 16, further comprising at least one additional sensor configured to be powered by the harvested energy and when so powered is configured to measure a parameter;
wherein the data transmitted comprises the parameter.

19. The apparatus of claim 18, wherein the parameter comprises pressure or acceleration.

20. The apparatus of claim 16, further comprising a smart metamaterial configured to exhibit a metamaterial characteristic under a select condition, and wherein the data transmitted comprises the metamaterial characteristic.

21. The apparatus of claim 7, wherein the data transmitted via NFC comprises manufacturing information, manufacturing date, lot number, trackability information and storage information.

22. The apparatus of any of claims 1-21, wherein the apparatus is comprised in a medicament device attachment component, said attachment component configured for attaching to a medicament device.

23. The apparatus of claim 22, wherein the attachment component comprises a rubber and/or silicone material, configured to attach to the medicament device to provide a grip for the medicament device.

24. An apparatus, comprising:
an antenna configured to harvest energy and when powered by harvested energy to convey the harvested energy;
an unpowered indicator configured to exhibit an excursion indicator only when a temperature excursion of a substance has occurred; and
at least one sensor configured to be powered by the harvested energy, wherein the at least one sensor comprises:
a registration sensor configured to be powered by the harvested energy and when so powered and when the excursion indicator is present, to generate a registration of the excursion indicator; and
a temperature sensor configured to be powered by the harvested energy and when so powered is configured to measure a current temperature of the substance;
wherein when powered by the harvested energy the apparatus is configured to transmit data comprising the registration of the excursion indicator and the current temperature of the substance.

25. The apparatus of claim 24, wherein the antenna comprises an NFC antenna configured to transmit data via NFC communication protocols.

26. The apparatus of claim 24 or 25, wherein the at least one sensor further comprises an injection sensor: configured to be powered by the harvested energy; configured when so powered to detect a wet injection; and configured to generate a registration of the wet injection when the wet injection has been detected; and wherein the data transmitted comprises the registration of the wet injection.

27. The apparatus of claim 24 or 25, wherein the at least one sensor further comprises a displacement sensor: configured to be powered by the harvested energy; configured when so powered to detect a displacement of the displacement sensor; and configured to generate a registration of the displacement when the displacement has been detected; and wherein the data transmitted comprises the registration of the displacement.

28. The apparatus of claim 24 or 25, wherein the at least one sensor further comprises an additional sensor configured to be powered by the harvested energy and when so powered is configured to measure a parameter; wherein the data transmitted comprises the parameter.

29. The apparatus of any of claims 24-28, comprising a delivery device comprising the Substance.

30. The apparatus of claim 29, wherein the delivery device comprises a medicament delivery device and the substance comprises a medicament.

31. The apparatus of claim 29, wherein the delivery device comprises a medicament delivery device simulator.

32. An apparatus, comprising:
an energy harvesting device configured to harvest energy and when powered by harvested energy to convey the harvested energy;
a communication device;
circuitry configured to be powered by the harvested energy and to be in data communication with the communication device;
an unpowered indicator configured to exhibit an excursion indicator only when a temperature excursion of a substance has occurred; and
a registration sensor configured to be powered by the harvested energy and when so powered and when the excursion indicator is present, to generate a registration of the excursion indicator;
wherein when powered by the harvested energy the circuitry is configured to transmit data comprising the registration of the excursion indicator via the communication device.

33. The apparatus of claim 32, further comprising an NFC antenna, wherein the NFC antenna is both the energy harvesting device and the communication device.

34. The apparatus of claim 32 or 33, wherein the energy harvesting device comprises a mechanical energy harvesting device.

35. The apparatus of any of claims 32-34, further comprising an NFC antenna, wherein the NFC antenna is the communication device.

36. The apparatus of any of claims 32-35, further comprising: a delivery device comprising the substance; and a mechanical activator configured: to dispense medicament via movement; or to simulate dispensing the substance via the movement; wherein the mechanical energy harvesting device harvests the energy from the movement of the mechanical activator.

37. The apparatus of claim 34, wherein the mechanical energy harvesting device is configured to harvest energy from movement of the mechanical energy harvesting device.

38. The apparatus of claim 32, wherein the energy harvesting device comprises a heat transfer energy harvesting device.

39. The apparatus of any of claims 32-38, wherein the unpowered indicator comprises a chemically activated sensor comprising a chemical that undergoes a change in chemical composition during the temperature excursion.

40. The apparatus of any of claims 32-38, wherein the unpowered indicator comprises a chemically activated sensor comprising a chemical that melts above a predetermined temperature associated with the temperature excursion.

41. The apparatus of any of claims 32-38, wherein the unpowered indicator comprises a chemically activated sensor comprising a chemical that undergoes an enzyme reaction above a predetermined temperature associated with the temperature excursion.

42. The apparatus of any of claims 32-38, wherein the unpowered indicator comprises a chemically activated sensor comprising a chemical that polymerizes above a predetermined temperature associated with the temperature excursion.

43. The apparatus of any of claims 32-38, wherein the unpowered indicator comprises a chemically activated sensor comprising a chemical that corrodes above a predetermined temperature associated with the temperature excursion.

44. The apparatus of any of claims 32-38, wherein the unpowered indicator comprises a chemically activated sensor comprising a chemical that forms liquid crystals above a predetermined temperature associated with the temperature excursion.

45. The apparatus of any of claims 32-38, wherein the unpowered indicator comprises a chemically activated sensor comprising a chemical that undergoes an irreversible microbiological change above a predetermined temperature associated with the temperature excursion.

46. The apparatus of any of claims 32-45, further comprising a temperature sensor configured to be powered by the harvested energy and when so powered is configured to measure a current temperature of the substance; wherein the data transmitted comprises the current temperature.

47. A method, comprising: removing a delivery device comprising a substance from cold storage; warming the substance; establishing communication with an energy harvesting substance event monitoring system associated with the delivery device by providing energy for an energy harvesting device in the energy harvesting substance event monitoring system; and capturing data from the energy harvesting substance event monitoring system, wherein the data comprises whether or not a passive sensor in the energy harvesting substance event monitoring system indicates a temperature excursion of the substance has occurred.

48. The method of claim 47, further comprising determining whether a temperature of the substance is at or above a predetermined temperature via a temperature sensor in the energy harvesting substance event monitoring system.

49. The method of claim 47 or 48, further comprising: inferring an activation of the delivery device at a date and a time the energy harvesting device harvests the energy, and determining a degree of adherence to an activation plan based at least in part on the inference.

50. The method of any of claims 47-49, further comprising using a smart device to at least one of: provide the energy; establish the communication; and to capture the data.

51. The method of claim 50, further comprising: using the smart device to establish the communication with the energy harvesting substance event monitoring system; and
reviewing training resources presented on the smart device once the communication with the energy harvesting substance event monitoring system is established.

52. The method of claim 50, further comprising: using the smart device to establish the communication with the energy harvesting substance event monitoring system; and
sharing the data from the energy harvesting substance event monitoring system with an authorized user via the smart device.

53. A method, comprising: maintaining a thermal association between a substance and an unpowered indicator, wherein a property of the unpowered indicator changes if the unpowered indicator exceeds a predetermined temperature; maintaining an association between a registration sensor and the unpowered indicator, wherein the registration sensor is configured to be powered by harvested energy and when so powered and when the excursion indicator is present, to generate a registration of the excursion indicator; and maintaining an association between an energy harvesting device and the registration sensor, wherein the energy harvesting device is configured to harvest energy and to deliver the harvested energy to the registration sensor when the harvesting device harvests energy.

54. The method of claim 53, further comprising: harvesting energy via the energy harvesting device; providing the harvested energy to the registration sensor; determining whether the excursion indicator is present via the registration sensor; and generating a registration of the excursion indicator via the registration sensor when the excursion indicator is present and when the registration sensor is powered by the energy harvesting device.

55. The method of claim 54, further comprising: establishing data communication between the registration sensor and a communication device configured to be powered by the harvested energy when the registration sensor and the communication device are powered by the harvested energy; and transmitting data via the communication device when the communication device is powered by the harvested energy, wherein the data comprises whether the registration is present.

56. The method of any of claims 53-55, further comprising: providing thermal communication between the substance and a temperature sensor that is configured to be powered by the harvested energy; measuring a temperature of the substance via the temperature sensor when the temperature sensor is powered by the harvested energy; and establishing data communication between the temperature sensor and the communication device; wherein the data comprises the temperature of the substance.

57. The method of any of claims 54-56, further comprising inferring an activation of a delivery device comprising the substance at a date and a time the energy harvesting device harvests the energy.

58. The method of claim 57, further comprising determining a degree of adherence to an activation plan based at least in part on the inference.

59. The method of claim 54, further comprising: delivering energy to the energy harvesting device via a smart device; and providing information via the smart device when the energy is delivered to the energy harvesting device, wherein the information comprises training materials related to the method.

60. The method of claim 53, further comprising: delivering energy to the energy harvesting device via a smart device; and sharing the data with an authorized user via the internet via the smart device.

61. The apparatus of any of claims 1-46, wherein the registration sensor comprises a photoresistor and/or color sensor.

62. The apparatus of any of claims 1-46 and 61, the circuitry comprises a light source and a light sensor, wherein the light source delivers light to the unpowered indicator which is reflected back to the light sensor, and wherein the reflected light changes based on a color change of the unpowered indicator such a change in the light is detectable by the light sensor.

63. The apparatus of claim 62, wherein the light source is a green light and the light sensor is a green light sensor, and wherein the unpowered indicator changes from white to red, or vice versa, based on an excursion of temperature, and wherein more light is reflected to the light sensor when the unpowered indicator is white versus red.
